(19) United States
(12) Patent Application Publication
(10) Pub. No.: US 2025/0295937 A1
(43) Pub. Date: Sep. 25, 2025

(54) METHODS OF IMPROVING PAIN WITH FOCUSED ULTRASOUND

(71) Applicant: West Virginia University Board of Governors on behalf of West Virginia University, Morgantown, WV (US)

(72) Inventors: Ali Rezai, Morgantown, WV (US); Victor Finomore, Morgantown, WV (US)

(21) Appl. No.: 19/083,892

(22) Filed: Mar. 19, 2025

Related U.S. Application Data
(60) Provisional application No. 63/567,569, filed on Mar. 20, 2024.

(51) Int. Cl. A61N 7/00

(52) U.S. Cl. CPC A61N 7/00; A61N 2007/0026

(57) ABSTRACT

A method of improving pain in a patient in need thereof is provided. The method can include delivering an ultrasound signal to a neural target site. The method can be performed without delivering microbubbles to open the blood brain barrier. The ultrasound signal can have a power of greater than zero watts and less than one hundred fifty watts. Methods also include triggering or exacerbating pain to activate neural activity in neural regions associated with the pain by exposing the patient to a pain-related and pain-specific cue.

### CROSS REFERENCE TO RELATED APPLICATIONS

This application claims priority to U.S. Provisional Application No. 63/567,569, filed on Mar. 20, 2024. The entirety of this application is hereby incorporated by reference for all purposes.

### TECHNICAL BACKGROUND

The present disclosure relates to delivering focused ultrasound to areas of a patient's nervous system to improve pain.

### BACKGROUND

Pain is generally defined as an unpleasant sensory and emotional experience associated with, or resembling that associated with, actual or potential tissue damage. When standard medicines and physical therapy fail to offer pain relief to a patient, surgical implants can be an option. Two types of implants to control pain are intrathecal drug delivery devices and spinal cord stimulators. Intrathecal drug delivery devices include a medicine pump connected to a catheter, which carries pain medicine from the pump to the intrathecal space around the spinal cord. Spinal cord stimulators deliver low-level electrical signals to the spinal cord or to specific nerves to block pain signals from reaching the brain. Both devices, however, are not ideal therapies for pain as they are invasive in nature. Transcutaneous electrical nerve stimulation (TENS) uses electrical stimulation to diminish pain. During a TENS procedure, low-voltage electrical current is delivered through electrodes that are placed on the skin near the source of pain to stimulate the nerves in the affected area. Although TENS is non-invasive, it is not effective for many common types of pain. As such, there is a need for methods and systems for improving pain that are non-invasive and effective for different types of pain.

### SUMMARY

In an aspect, a method of improving pain in a patient in need thereof is provided. Such a method comprises delivering an ultrasound signal to a neural target site without delivering microbubbles to open the blood brain barrier. The neural target site can include a somatosensory cortex, a motor cortex, an association cortex, a posterior insula, a thalamus, a site of the dorsal column medial lemniscus pathway, a periaqueductal gray, a periventricular gray, an amygdala, a ventral striatum, a nucleus accumbens, an anterior insula, an anterior cingulate cortex, a dorsolateral prefrontal cortex, an orbitofrontal cortex, a caudate nucleus, a spinothalamic tract, a trigeminal ganglion, a trigeminal nucleus, a trigeminothalamic tract, a site of the peripheral nervous system, or combinations thereof. The ultrasound signal can comprise a power of greater than zero watts and less than one hundred fifty watts.

In another aspect, a feedback method of improving pain is provided. Such a method can comprise obtaining a measurement of a baseline value of a parameter associated with the patient's pain, applying an initial ultrasound signal to a neural target site of the patient, obtaining a subsequent measurement of a resultant value of the parameter associated with the patient's pain during or after application of the initial ultrasound signal, obtaining a comparison of the resultant value to the baseline value, and adjusting application of a subsequent ultrasound signal to the neural target site based on the comparison of the baseline value and the resultant value. The parameter can be a physiological, a cognitive, a motor/musculoskeletal, a sensory, a sleep, a psychosocial/behavioral parameter, a patient's response to a pain scale, or combinations thereof.

### BRIEF DESCRIPTION OF THE DRAWINGS

FIG. 1 is a flow chart of a method of improving pain according to an aspect of the present invention.

FIG. 2 is a flow chart of a method of improving pain according to an aspect of the present invention.

### DETAILED DESCRIPTION

The present disclosure relates to methods of improving pain. As used herein with respect to a described element, the terms “a,” “an,” and “the” include at least one or more of the described element(s) including combinations thereof unless otherwise indicated. Further, the terms “or” and “and” refer to “and/or” and combinations thereof unless otherwise indicated. By “substantially,” “approximately, or “about” is meant that the shape, size, configuration or value of the described element need not have the mathematically exact described shape, size, configuration or value of the described element but can have a shape, size, configuration or value that is recognizable by one skilled in the art as generally or approximately having the described shape, size, configuration, or value of the described element. As such “substantially,” “approximately,” or “about” refers to the complete or nearly complete extent of a characteristic, property, state, structure or value. The exact allowable degree of deviation from the characteristic, property, state, structure, or value will be so as to have the same overall result as if the absolute characteristic, property, state, structure, or value were obtained. The terms “first,” “second,” etc. are used to distinguish one element from another and not used in a quantitative sense unless indicated otherwise. Thus, a “first” element described below could also be termed a “second” element. A “patient” as used herein is a human being.

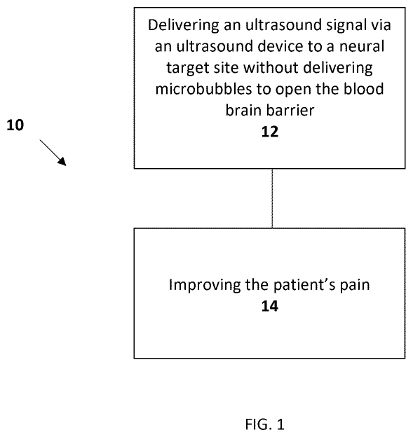

The present disclosure relates to improving pain in a patient who is suffering therefrom by using focused ultrasound therapy (e.g. delivering an ultrasound signal to the patient). By improving pain, the patient's pain is less severe after ultrasound therapy than before ultrasound therapy. In an aspect, and with reference to FIG. 1, a method 10 of improving pain in a patient in need thereof is provided. The method can include delivering an ultrasound signal via an ultrasound device to a target site of the patient's nervous system 12 (a neural target site) without delivering microbubbles to open the blood brain barrier. To open/disrupt the blood brain barrier, microbubbles are generally introduced into the brain and an ultrasound signal is delivered. The soundwaves of such a signal cavitate the microbubbles so that the microbubbles expand, which opens the blood brain barrier. Non-limiting neural target site are listed in Table I below. The method can further comprise improving the patient's pain 14.

TABLE I
Sensory-Discriminative Networks and Brain Targets
Somatosensory Cortex (S1/S2)
Motor Cortex
Association Cortex
Posterior Insula
Sensor Nucleus of the Thalamus
Intralaminar Nucleus of the Thalamus
Medial Lemniscus of the Dorsal Column Medial Lemniscus Pathway
Periaqueductal Gray
Periventricular Gray
Affective-Motivational Networks and Brain Targets
Amygdala
Ventral Striatum
Nucleus Accumbens
Anterior Insula
Cingulate Cortex
Cognitive-Executive Function Networks
Dorsolateral Prefrontal Cortex
Orbitofrontal Cortex
Periaqueductal Gray
Periventricular Gray
Caudate Nucleus
Peripheral Nervous System Targets
Dorsal Root Ganglion
Autonomic Target Sites (Sympathetic/Parasympathetic
Sites/Ganglions)
stellate ganglion
lumbar sympathetic ganglion
sphenopalatine ganglion
Celiac Plexus
Splanchnic Plexus/Nerve
Superior Hypogastric Plexus
Ganglion Impar
Other Neural Target Sites including Spinal Cord
Dorsal Column of the Dorsal Column Medial Lemniscus Pathway
Spinothalamic Tract
Trigeminal Nucleus
Trigeminal Ganglion
Trigeminothalamic Tract

With respect to parameters of the ultrasound signal, the signal can be delivered to the neural target site at a power of greater than zero watts and less than one hundred fifty watts. In certain aspects, the power is between fifty watts and one hundred watts. The ultrasound therapy can be delivered for a suitable time period such as, for example, greater than zero minutes to up to about thirty minutes. The ultrasound therapy can also be delivered at a suitable frequency such as, for example, at a frequency of 0.1 MHz to 0.3 MHz. In certain aspects, the frequency is between 0.1 MHz and 0.275 MHz.

Other non-limiting parameters of the ultrasound signal are sonication dose, power (e.g. less than 150 W), sonication duration (e.g. 0 min-60 min), frequency direction, repetition time On/Off (e.g. 5 sec; 10 sec), pulse duration on/off (e.g. l00 msec; 900 msec), continuous or intermittent, energy/minute (e.g. 0 J/min-290 J/min, and frequency (e.g. 0.1-3 MHz), number of elements (e.g. 1-1024), and waveshape form.

In one aspect, focused ultrasound therapy is provided with a power between 60 W and 90 W. In another aspect, focused ultrasound therapy is provided with a power between 70 W and 100 W. In a further aspect, focused ultrasound therapy is provided with a power between 40 W and 70 W. In a further aspect, focused ultrasound therapy is provided with a power greater than zero and less than 70 W. In a further aspect, focused ultrasound therapy is provided with a power greater than zero and less than 100 W. In a further aspect, focused ultrasound therapy is provided with a power less than 150 W. In a further aspect, focused ultrasound therapy is provided with a power between 40 W and 100 W. In a further aspect, focused ultrasound therapy is provided with a power between 50 W and 90 W. In a further aspect, focused ultrasound therapy is provided with a power between 60 W and 100 W. In a further aspect, focused ultrasound therapy is provided with a power between 70 W and 120 W.

In one aspect, focused ultrasound therapy is provided with a pulse width between 10 ms and 100 ms. In another aspect, focused ultrasound therapy is provided with a pulse width between 10 ms and 50 ms. In a further aspect, focused ultrasound therapy is provided with a pulse width between 10 ms and 200 ms. In a further aspect, focused ultrasound therapy is provided with a pulse width between 50 ms and 200 ms. In a further aspect, focused ultrasound therapy is provided with a pulse width between 100 ms and 200 ms.

In one aspect, a session of focused ultrasound therapy lasts between three minutes and seven minutes. In another aspect, a session of focused ultrasound therapy lasts between five minutes and twenty minutes. In a further aspect, a session of focused ultrasound therapy lasts between three minutes and ten minutes. In a further aspect, a session of focused ultrasound therapy lasts between five minutes and twenty minutes. In a further aspect, a session of focused ultrasound therapy lasts between ten minutes and twenty minutes. In a further aspect, a session of focused ultrasound therapy lasts between ten minutes and thirty minutes. In a further aspect, a session of focused ultrasound therapy lasts between ten minutes and sixty minutes. In one aspect, focused ultrasound therapy is provided with a frequency between 0.02 MHz and 0.1 MHz. In another aspect, focused ultrasound therapy is provided with a frequency between 0.2 MHz and 0.3 MHz. In a further aspect, focused ultrasound therapy is provided with a frequency between 0.4 MHz and 0.6 MHz. In a further aspect, focused ultrasound therapy is provided with a frequency between 0.4 MHz and 0.8 MHz. In a further aspect, focused ultrasound therapy is provided with a frequency between 0.7 MHz and 1 MHz. In a further aspect, focused ultrasound therapy is provided with a frequency between 1 MHz and 2 MHz. In a further aspect, focused ultrasound therapy is provided with a frequency between 2 MHz and 3 MHz. In a further aspect, focused ultrasound therapy is provided with a frequency between 0.1 MHz and 3 MHz. In certain aspects, the pulse duration is about 100 ms. In certain aspects, the acoustic pressure is between about 0.1 MPa and about 2.5 MPa. In certain aspects, the acoustic pressure is about 2 MPa. In certain aspects, the mechanical index is between about 0.1 and about 5. In certain aspects, the mechanical index is 4.

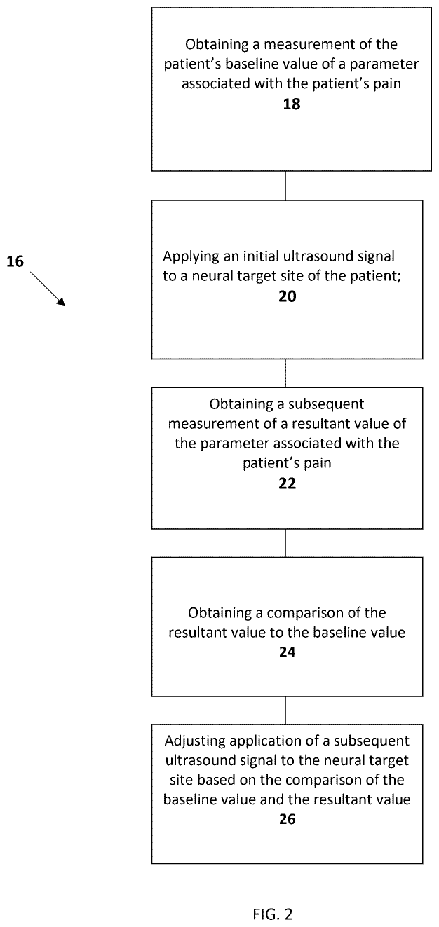

In certain aspects, a methods of improving pain incorporates objective and/or subjective feedback (e.g. qualitative and/or quantitative measurements) to adjust ultrasound therapy. Such feedback can aid the clinician in monitoring the patient's status and condition and to provide or adjust ultrasound therapy accordingly as well as providing preemptive ultrasound therapy. In reference to FIG. 2, a method 16 comprises obtaining a measurement of a patient's baseline value (which can be taken at any suitable point(s) before, during or after a given therapy session) of a parameter associated with the patient's pain 18, applying an initial ultrasound signal to a neural targe site of the patient 20, obtaining a subsequent measurement of a resultant value of the parameter associated with patient's pain 22, obtaining a comparison of the resultant value to the baseline value 24 and adjusting application of a subsequent ultrasound signal to the neural target site based on the comparison of the baseline value and the resultant value 26. The ultrasound therapy can be provided without delivering microbubbles to open the blood brain barrier as discussed above and can include the neural sites discussed above. The parameter associated with the patient's pain can be a physiological parameter, a cognitive parameter, a motor/musculoskeletal parameter, a sensory parameter, a sleep parameter, and/or a psychosocial/behavioral parameter.

Table I provides non-limiting examples of physiological parameters that can be measured and exemplary tests, devices, and methods, to measure the physiological parameters.

TABLE I

| | Exemplary Devices and Methods to |
|---|---|
| Physiological Parameter | Measure Physiological Parameters |
| Brain Activity | Electroencephalogram, Magnetic |
| | Resonance Imaging, including |
| | functional Magnetic Resonance Imaging |
| | (fMRI), PET, SPECT, MEG, |
| | near-infrared spectroscopy, functional |
| | near-infrared spectroscopy, and other |
| | brain imaging modalities looking at |
| | electrical, blood flow, |
| | neurotransmitter, and metabolic MRI, |
| | taken either during a cognitive task or |
| | while the patient is at rest |
| Brain Structure | Magnetic Resonance Imaging |
| Heart rate | Electrocardiogram and |
| | Photoplethysmogram |
| Heart rate variability | Electrocardiogram, |
| | Photoplethysmogram |
| Eye tracking | Pupillometry, including tracking |
| | saccades, fixations, and pupil size |
| | (e.g., dilation) |
| Perspiration | Perspiration sensor |
| Retinal | OCT and angiography |
| Retinal anatomy, layers, retinal | Other Retinal imaging including wide |
| vasculature | field |
| Blood pressure | Sphygmomanometer |
| Body temperature | Thermometer, infrared thermography |
| Blood oxygen saturation | Pulse oximeter, Near-infrared |
| | spectroscopy, functional near-infrared |
| | specroscpy |
| respiratory rate | Pulse oximeter, photopethysmogram, |
| | capaciflector, respiratory belt |
| Skin conductivity | Electrodermal activity |
| Facial emotions | Camera or EMG based sensors for |
| | emotion and wellness |
| Sympathetic and | Derived from the above measurements |
| parasympathetic tone | |

In certain aspects a physiological parameter can be measured using imaging techniques. Measuring an imaging parameter can comprise assessing a change identified in a PET scan, a change in resting state functional MRI, a change in task-based MRI, a change in brain networks and neural function, a change in a default mode neural network to salience and executive neural networks, or combinations thereof. A physiological parameter can be an autonomic parameter and measuring such an autonomic parameter can comprise, for example, assessing a change in skin color or body temperature, or the onset or increase of swelling of the patient.

The physiological parameters can be measured in clinical settings with appropriate devices or in non-clinical settings via wearable, implantable, or portable devices. Some information can also be determined from self-reporting by the user via applications in a mobile device or via interaction with applications on the mobile device. For example, a smart watch, ring, or patch can be used to measure the user's heart rate, heart rate variability, body temperature, blood oxygen saturation, movement, and sleep. In a non-clinical setting, these values can also be subject to a diurnal analysis to estimate variability. Eye tracking can be performed, for example, using a camera on a mobile device and specialized software.

Table II provides non-limiting examples of cognitive parameters that can be measured and exemplary methods and tests/tasks to measure such cognitive parameters.

TABLE II

| | Exemplary Tests and Methods to |
|---|---|
| Cognitive Parameter | Measure Cognitive Parameters |
| Temporal discounting | Kirby Delay Discounting Task |
| Alertness and fatigue | Psychomotor Vigilance Task |
| Focused attention and response | Erikson Flanker Task |
| inhibition | |
| Working memory | N-Back Task |
| Attentional bias towards | Dot-Probe Task |
| emotional cues | |
| Inflexible persistence | Wisconsin Card Sorting Task |
| Decision making | Iowa Gambling Task |
| Risk taking behavior | Balloon Analogue Risk Task |
| Inhibitory control | Anti-Saccade Task |
| Sustained attention | Sustained Attention |
| Executive function | Task Shifting or Set Shifting Task |
| Long term memory | Identifying pictures of famous people |
| | and other memory related tasks |

These cognitive tests can be administered in a clinical/laboratory setting or in a naturalistic, non-clinical setting such as when the user is at home, work or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch. In one example, the patient can be allowed to explore a virtual reality environment and collect items within the environment. The patient is then asked to recount where each item was found within the virtual environment and the relationship of that location to a starting point, testing the patient's ability to recall spatial relationships among the virtual locations.

TABLE III provides non-limiting examples of parameters associated with movement and activity of the user, referred to herein alternatively for ease of reference as “motor/musculoskeletal parameters,” that can be measured and exemplary tests, devices, and methods. The use of portable monitoring, physiological sensing, and portable computing devices allows the motor/musculoskeletal parameters to be measured. Using embedded accelerometer, GPS, and cameras, the user's movements can be captured and quantified. Range of motion and gait analysis can be performed in a clinical setting using appropriate motion capture and camera equipment for evaluation.

TABLE III

| Motor/Musculoskeletal | Exemplary Tests and Methods to Measure |
|---|---|
| Parameter | Motor/Musculoskeletal Parameters |
| Activity level | Daily movement total, time of activities, from |
| | wearable accelerometer, steps, Motion Capture |
| | data, gait analysis, GPS, deviation from |
| | established geolocation patterns, force plates |
| Gait analysis | Gait mat, camera, force plats |
| Range of motion | Motion capture, camera |

TABLE IV provides non-limiting examples of parameters associated with sensory acuity of the user, referred to herein alternatively for ease of reference as “sensory parameters,” that can be measured and exemplary tests, devices, and methods.

TABLE IV

| | Exemplary Tests and Methods to Measure sensory |
|---|---|
| Sensory Parameter | Parameters |
| Vision | Visual acuity test, visual field tests, eye tracking, |
| | EMG, presentation of lights (i.e. flashes, intensity, |
| | colors) |
| Hearing | Hearing tests, presentation of sounds (i.e. intensity, |
| | tones) |
| Touch | Two-point discrimination, frey filament, hot/cold |
| | press, pressure |
| Smell/taste | Presentation of odors or flavors |
| Vestibular | Vestibula function test |

TABLE V provides non-limiting examples of parameters associated with a sleep quantity, phases, and quality of the user, referred to herein alternatively for ease of reference as “sleep parameters,” that can be measured and exemplary tests, devices, and methods.

TABLE V

| | Exemplary Tests and Methods to Measure Sleep |
|---|---|
| Sleep Parameter | Parameters |
| Sleep from wearables | Sleep onset & offset, sleep quality, sleep quantity, |
| | from wearable accelerometer, temperature, and |
| | PPG, |
| Sleep Questions | Pittsburg Sleep Quality Index, Functional |
| | Outcomes of Sleep Questionnaire, Fatigue |
| | Severity Scale, Epworth Sleepiness Scale |
| Devices | Polysomnography; ultrasound, camera, bed |
| | sensors, EEG |
| Circadian Rhythm | Light sensors, actigraphy, serum levels, core body |
| | temperature |

Table VI provides non-limiting examples of psychosocial and behavioral parameters that can be measured and exemplary tests, devices, and methods.

TABLE VI

| Psychosocial or Behavioral | Exemplary Tests and Methods to Measure |
|---|---|
| Parameter | Psychosocial or Behavioral Parameters |
| Pain scales | visual analog scales, pain diary, pain body |
| | map |
| Medical Records | Medical history, prescriptions, setting for |
| | treatment devices such as spinal cord |
| | stimulator, imaging data |
| Wellness Rating | Visual Analog Scale, Defense & Veterans |
| | wellness rating scale, wellness scale, |
| | Wellness Assessment screening tool and |
| | outcomes registry |
| Burnout | Burnout inventory or similar |
| Physical, Mental, and Social | User-Reported Outcomes Measurement |
| Health | Information System (PROMIS), Quality of |
| | Life Questionnaire |
| Depression | Hamilton Depression Rating Scale |
| Anxiety | Hamilton Anxiety Rating Scale |
| Mania | Snaith-Hamilton Pleasure Scale |
| Mood/ | Profile of Mood States; Positive Affect |
| Catastrophizing scale | Negative Affect Schedule |
| Affect | Positive Affect Negative Affect Schedule |
| Impulsivity | Barratt Impulsiveness Scale |
| Adverse Childhood | Childhood trauma |
| Experiences | |
| Daily Activities | Exposure, risk taking |
| Daily Workload and Stress | NASA Task Load Index, Perceived Stress |
| | Scale (PSS), Social Readjustment Rating |
| | Scale (SRRS) |
| Social Determents of Health | Social determents of health questionnaire |

The behavioral and psychosocial parameters can measure the user's functionality as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the facial expression analysis to analyze the user's facial expressions that could indicate mood, anxiety, depression, agitation, and fatigue. This affect detection can be performed using an appropriate predictive model trained on faces of users mimicking or experiencing a given emotion or mental state.

In addition or in alternative to the parameters described above, another parameter to assess a patient's baseline pain level (baseline value) and/or subsequent pain level (resultant pain value) can be the patient's response to a pain scale. Various pain scales can be used such as a Numerical Rating Scale (NRS), a Visual Analog Scale (VAS), categorical scales, or combinations thereof. An NRS uses numbers for a patient to rate pain; a VAS asks a patient to select a picture that best matches the patient's pain level; and a categorical scale asks a patient to primarily uses words, possibly along with numbers, colors, or location(s) on the body to gauge the patient's pain. Non-limiting examples of pain scales include the Wong-Baker Faces Pain Scale, the FLACC Pain Scale, the CRIES Pain Scale, the COMFORT Pain Scale, the McGill Pain Questionnaire, the Color Analog Pain Scale, the Mankoski Pain Scale, the Brief Pain Inventory, the Descriptor Differential Scale of Pain Intensity, and the Defense and Veterans Pain Rating Scale. Such pain scales are only exemplary and other pain scales or quantitative and/or qualitative tests can be used to assess the patient's pain level.

In certain aspects, the patient can be exposed to a cue associated with the patient's pain before or during application of the initial ultrasound signal to initiate, activate, trigger or exacerbate pain in the patient prior to or concurrent with ultrasound therapy (as well as to adjust ultrasound therapy and select a patient as a viable candidate for ultrasound therapy). In certain aspects, such cues can effectively serve to “prime” the patient for therapy to activate/increase neural activity in neural regions associated with the patient's pain. The cue can be a visual cue, a gustatory cue, an auditory cue, a tactile cue, an olfactory cue, or combinations thereof that is known, for example, to trigger/exacerbate the patient's pain. The cue(s) can be specific for the particular type of pain for which the patient is seeking therapy. For example, if the patient is suffering from postherpetic neuralgia, the cue can be a tactile cue such as a mild burning pain stimulus and/or a visual cue such as a rash or blisters (e.g. associated with shingles). If the patient is suffering from neuropathic pain, the cue can also be a tactile cue such as a stimulus that causes mild numbness, prickling, tingling, or a burning sensation. If the patient is suffering from allodynia, hyperalgesia, or otherwise extreme sensitivity to pain signals, the patient can be exposed to a tactile stimulus or a change in temperature, for example. If the patient is suffering from a migraine, the cue can be a trigger that causes the patient to experience a migraine such as an olfactory cue or auditory cue such as particular smells or sounds. The above examples are only exemplary and are meant to point out that the cues can be pain specific and can stimulate different senses. In addition or in alternative to the cue eliciting an acute pain response, the cue can also evoke a physiological, cognitive, emotional, psychosocial and/or behavioral response. Non-limiting examples of such responses include anxiety, fear, mood, energy attention, skin discoloration, body temperatures, swelling or combinations thereof. The patient can be exposed to multiple cues, including multiple different types of cues during any assessment period. Further, the patient can be exposed to the cues via a smart phone, tablet, personal computer or laptop, for example, in a naturalistic non-clinical setting such as when the patient is at home, work or other non-clinical setting. The patient also can be exposed to the cues via virtual reality, augmented reality, or mixed reality.

The type of change to the patient's pain level (the resultant value) can influence whether ultrasound therapy is provided or if existing ultrasound therapy should be adjusted. For example, if there is an increase in the patient's pain level compared to the baseline pain level, a method can involve initiating ultrasound therapy. Conversely, if the pain level is substantially the same as the baseline pain level, ultrasound therapy may not be applied. In terms of adjusting ultrasound therapy, methods can involve adjusting the parameters or dosing of the ultrasound therapy such as, for example, the duration, power, or frequency of the ultrasound therapy and/or changing the neural target site. If there is an increase in the patient's resultant pain level compared to the baseline pain level, a method can involve adjusting the ultrasound therapy so that the ultrasound therapy is more effective. For example, if the patient was previously having ultrasound therapy delivered for five minutes during a therapy session, the patient can have the ultrasound therapy subsequently delivered for twenty minutes during each session or if the patient was having ultrasound therapy delivered every thirty days, the patient can have ultrasound therapy subsequently delivered every two weeks. Conversely, if the resultant pain level is substantially the same as the baseline pain level, the ultrasound parameters may not need adjustment and subsequent ultrasound sessions can serve primarily as maintenance sessions or the intensity, frequency or duration of the ultrasound therapy can be decreased, for example. Alternatively, if the resultant pain level during is substantially the same as the baseline pain level, then the patient can stop receiving any subsequent ultrasound therapy. The above scenarios are only exemplary and are provided to illustrate that the presence and type of change of the patient's pain level can influence whether ultrasound therapy is provided or if existing ultrasound therapy should be adjusted or terminated.

Further, the degree of the pain level can influence the parameters of initial or subsequent ultrasound therapy. For example, if the specific patient seeking therapy has a pain level that is higher than the average pain level of the same patient population (patients with the same type of pain), the ultrasound therapy can be more aggressive initially or subsequently (e.g. the duration, frequency, or power of the ultrasound therapy can be greater than that provided to patients of the same patient population). Similarly, if the specific patient seeking therapy has a pain level that is higher than the average pain level of the same patient population, the ultrasound therapy can be more aggressive initially or subsequently. Conversely, if the specific patient's pain level is lower than the average pain level of the same patient population, the ultrasound therapy can be less aggressive initially or subsequently. In other words, the severity or degree of the patient's resultant pain level (as well as baseline levels) can correlate to the degree or aggressiveness of the ultrasound therapy. The above scenarios are only exemplary and are provided to illustrate that the degree of change of the patient's pain levels can influence the parameters of initial and subsequent ultrasound therapy.

In addition to the above-referenced feedback parameters, other data can be part of a multi-dimensional feedback approach to improving pain including clinical data. Such clinical data can include, for example, the patient's clinical state, the patient's medical history (including family history), employment information, and residential status.

Once ultrasound therapy has been provided, feedback (types of which are discussed above) can be collected to determine if the treatment has been effective. This can be done during or immediately after treatment (“acute feedback”), a short time (e.g. within a month such as, for example, five hours to five days) after a treatment (“subacute feedback”) or a longer time (e.g., more than five days after a treatment or more than a month after treatment (“chronic feedback”).

The pain the patient is suffering from can be chronic pain and can have various etiologies. Non-limiting examples of pain include postherpetic neuralgia, neuropathic pain (including neuropathic facial pain, coccygodynia, stroke pain, nociceptive pain, central pain, complex regional pain syndrome, autonomic pain, postamputation pain, cancer pain, abdominal pain (including chronic pancreatitis or malignancies of abdominal origin such as e.g. pancreatic cancer), endometriosis, pelvic pain, post-surgical pain, headaches (including cluster headaches and/or migraine headaches) or combinations thereof. In aspects where the pain is headache such as, for example, a migraine headache and/or a cluster headache, the neural target site can be a sphenopalatine ganglion.

The disclosed methods can be used to initially treat a patient, screen or select a patient for therapy, and adjust follow up ultrasound therapy sessions. Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. A method of improving pain in a patient in need thereof comprising:
delivering an ultrasound signal to a neural target site without delivering microbubbles to open the blood brain barrier, the neural target site comprising a somatosensory cortex, a motor cortex, an association cortex, a posterior insula, a thalamus, a site of the dorsal column medial lemniscus pathway, a periaqueductal gray, a periventricular gray, an amygdala, a ventral striatum, a nucleus accumbens, an anterior insula, an anterior cingulate cortex, a dorsolateral prefrontal cortex, an orbitofrontal cortex, a caudate nucleus, a spinothalamic tract, a trigeminal ganglion, a trigeminal nucleus, a trigeminothalamic tract, a site of the peripheral nervous system, or combinations thereof, the ultrasound signal having a mechanical index of between about 1.0 and 5.0 and/or an acoustic pressure of between about 1.0 MPa to about 2.5 MPa improving the patient's pain.

2. The method of claim 1, wherein the neural target site is the somatosensory cortex, the motor cortex, the association cortex, the posterior insula, a sensor nucleus of the thalamus, an intralaminar nucleus of the thalamus, a medial lemniscus of the dorsal column medial lemniscus pathway, the periaqueductal gray, or the periventricular gray.

3. The method of claim 1, wherein the neural target site is the amygdala, the ventral striatum, the nucleus accumbens, the anterior insula, or the anterior cingulate cortex.

4. The method of claim 1, wherein the neural target site is the dorsolateral prefrontal cortex, the orbitofrontal cortex, or the caudate nucleus.

5. The method of claim 1, wherein the neural target site is a dorsal column of the dorsal column medial lemniscus pathway, the spinothalamic tract, the trigeminal nucleus, the trigeminal ganglion, or the trigeminothalamic tract.

6. The method of claim 1, wherein the site of the peripheral nervous system is a dorsal root ganglion, a stellate ganglion, a lumbar sympathetic ganglion, a sphenopalatine ganglion, a celiac plexus, a splanchnic nerve, a splanchnic plexus, a superior hypogastric plexus, or a ganglion Impar.

7. The method of claim 1, wherein the power is between fifty watts and one hundred watts.

8. The method of claim 1, wherein the pain is chronic pain.

9. The method of claim 1, wherein the pain is postherpetic neuralgia, neuropathic pain, coccygodynia, stroke pain, nociceptive pain, central pain, complex regional pain syndrome, postamputation pain, cancer pain, abdominal pain, post-surgical pain, autonomic pain, endometriosis, pelvic pain, a headache, or combinations thereof.

10. The method of claim 9, wherein the neuropathic pain is neuropathic facial pain.

11. The method of claim 9, wherein the pain is a headache.

12. The method of claim 11, wherein the headache is a migraine headache.

13. The method of claim 11, wherein the headache is a cluster headache.

14. A method of improving pain in a patient in need thereof comprising:
obtaining a measurement of a baseline value of a parameter associated with the patient's pain comprising a physiological, a cognitive, a motor/musculoskeletal, a sensory, a sleep, a psychosocial/behavioral parameter, a patient's response to a pain scale, or combinations thereof;
applying an initial ultrasound signal to a neural target site of the patient;
obtaining a subsequent measurement of a resultant value of the parameter associated with the patient's pain during or after application of the initial ultrasound signal;
obtaining a comparison of the resultant value to the baseline value; and
adjusting application of a subsequent ultrasound signal to the neural target site based on the comparison of the baseline value and the resultant value.

15. The method of claim 14, further comprising exposing the patient to a cue associated with the patient's pain before or during applying the initial ultrasound signal to trigger or exacerbate the patient's pain.

16. The method of claim 15, wherein the cue is a visual cue, a gustatory cue, an auditory cue, a tactile cue or stimulus, an olfactory cue, or combinations thereof.

17. The method of claim 15, wherein exposure to the cue evokes a physiological, cognitive, emotional, psychosocial and/or behavioral response.

18. The method of claim 15, wherein the resultant value is assessed during exposure to the cue, within a month after the initial or subsequent ultrasound application, a month or more after the initial or subsequent ultrasound application, or combinations thereof.

19. The method of claim 14, wherein the neural target site is a somatosensory cortex, a motor cortex, an association cortex, a posterior insula, a thalamus, a site of the dorsal column medial lemniscus pathway, a periaqueductal gray, a periventricular gray, an amygdala, a ventral striatum, a nucleus accumbens, an anterior insula, an anterior cingulate cortex, a dorsolateral prefrontal cortex, an orbitofrontal cortex, a caudate nucleus, a spinothalamic tract, a trigeminal ganglion, a trigeminal nucleus, a trigeminothalamic tract, a site of the peripheral nervous system, or combinations thereof.

20. The method of claim 14, wherein applying or adjusting application comprises providing an ultrasound signal to the neural target site without delivering microbubbles to open the blood brain barrier.

21. The method of claim 14, wherein the pain is chronic pain.

22. The method of claim 14, wherein the pain is postherpetic neuralgia, neuropathic pain, coccygodynia, stroke pain, nociceptive pain, central pain, complex regional pain syndrome, postamputation pain, cancer pain, abdominal pain, post-surgical pain, autonomic pain, endometriosis, pelvic pain, a headache, or combinations thereof.

23. The method of claim 22, wherein the neuropathic pain is neuropathic facial pain.

24. The method of claim 22, wherein the pain is a headache.

25. The method of claim 24, wherein the headache is a migraine headache.

26. The method of claim 24, wherein the headache is a cluster headache.

27. The method of claim 14, wherein adjusting application of the subsequent ultrasound signal comprises increasing the frequency, duration, intensity, or a combination thereof of the ultrasound signal or changing a neural target site of the ultrasound signal upon a determination that the resultant value increases above the patient's baseline value.

28. The method of claim 14, wherein adjusting application of the subsequent ultrasound signal comprises decreasing the frequency, duration, intensity, or a combination thereof of the ultrasound signal; maintaining the same frequency, duration, intensity or a combination thereof of the ultrasound signal; or maintaining a neural target site of the ultrasound signal ultrasound upon a determination that the resultant value remains substantially the same as the patient's baseline value.

29. The method of claim 14 obtaining a comparison of the resultant value to the baseline value comprises assessing a change identified in a PET scan, a change in resting state functional MRI, a change in task based MRI, a change in brain networks and neural function, a change in a default mode neural network to salience and executive neural networks, or combinations thereof.

30. The method of claim 14, wherein obtaining a comparison of the resultant value to the baseline value comprises assessing a change in skin color or body temperature, or the onset or increase of swelling of the patient.
